(19) United States
(12) Patent Application Publication
(10) Pub. No.: US 2017/0231834 A1
(43) Pub. Date: Aug. 17, 2017

(54) Disposable Underwear

(71) Applicant: Jasmine Sang, Royal Palm Beach, FL (US)

(72) Inventor: Jasmine Sang, Royal Palm Beach, FL (US)

(21) Appl. No.: 15/044,080

(22) Filed: Feb. 15, 2016

(51) Int. Cl. A61F 13/47; A61F 13/56; A61F 13/476; A61F 13/53; A61F 13/496

(52) U.S. Cl. CPC A61F 13/47; A61F 13/53; A61F 13/496; A61F 13/476; A61F 13/5605; A61F 2013/16

(57) ABSTRACT

The present invention relates to wearable sanitary napkin/panty liner having wearable section further comprising an upper side, a lower side a front surface and an inner surface, pocket located at the inner surface of the wearable section, waist band located at the upper side of the wearable section and sanitary napkin is placed within the pocket located at the inner surface of the wearable section and the pocket is permanently sealed.

### CROSS REFERENCE TO RELATED APPLICATIONS

Not applicable

### FEDERALLY SPONSORED RESEARCH AND DEVELOPMENT

Not applicable.

### MICROFICHE Not applicable

### BACKGROUND OF THE INVENTION

(1) Field of the Invention

The present invention relates generally to clothing and more particularly clothing suitable for use by women who desire to protect against staining or leakage from the flow of bodily fluids, The invention is, however, even more particularly relates to a disposable and comfortable undergarment that has a sanitary pad into the undergarment.

(2) Background of the Invention

Sanitary napkins are absorbent devices designed to receive and contain vaginal discharges, such as menses, spotting, and leaking bladders. They are omnipresent items of modern society. In recent periods disposable sanitary napkins designed to be held adjacent to the human body through the agency of a garment, such as a disposable wearable sanitary napkin/panty liner, having a fixed sanitary napkin.

In recent years, in part as a result of these problems, sanitary napkins have evolved to be thinner and more flexible. For example, some sanitary napkins have wings to add attachment to the underwear and help prevent soiling, and others are provided with different colors to match clothing. Sanitary napkins are also available in various shapes and sizes, e.g., even in a shape to fit thong style underwear. These sanitary napkins are generally triangular in shape, essentially matched to the triangular shape of the front/bottom area of the thong style underwear, wherein the sides of the triangle typically extend out to the thighs. However, such sanitary napkins move around or require some mechanism to attach the sanitary napkin to the underwear, e.g., an adhesive backing or extending wing structures for wrapping around the underwear.

Sanitary pads used with regular undergarments often leak through and move around in the undergarment. When women go to sleep with the regular sanitary pad and undergarment, the sheets usually get stained, or when sitting in a chair, the chair will often get stained. Skirts, dresses and pants get stained because of the insecurity of the regular sanitary pad and undergarment.

Disposable undergarments are very well known in the art. Most are directed toward hygienic panties having absorbent pads for menstrual use, incontinence, etc. The most disposable undergarments of the aforementioned type are bulky and require a large box or bag for carrying and are not lightweight or comfortable or otherwise suitable for everyday use such as illustrated in U.S. Pat. App. No. US 20080132870 A1, U.S. Pat. App. No. US 20050043702 A1, U.S. Pat. App. No. US 20140378936 A1 and U.S. Pat. App. No. US 20120232516 A1.

U.S. Patent application no. US 20080132870 A1 filed by Edith Nelson shows a combined disposable panty and sanitary pad undergarment includes a panty body having a front part, a rear part forming in combination with the front part a top edge of the panty body, and a crotch part joining the rear and front part and forming a pair of leg openings. An absorption pad is overlaid over and secured to the crotch part and a lower portion of each front and rear part.

U.S. Patent application no. US 20050043702 A1 filed by Kathy Houchen & Rhonda Smith shows a disposable undergarment comprises a front portion made of disposable material, a rear portion made of disposable material, and a crotch portion made of disposable material. The crotch portion includes a sanitary pad fixed to the undergarment in a pocket or pouch as part of the undergarment.

U.S. Patent application no. US 20140378936 A1 filed by Tailored Technologies, Inc. shows an undergarment including a pocket extending from an anterior crotch portion of the undergarment to a posterior crotch portion of the undergarment, the pocket closed along three sides and opening in a direction of the anterior crotch, a hood arranged on the anterior crotch portion concealing the opening of the pocket, and an absorbent pad removable from the pocket, wherein the hood is configured to be pulled aside to expose the opening of the pocket to remove and insert the absorbent pad from the pocket.

U.S. Patent application no. US 20120232516 A filed by Albania Leon shows a disposable padded underwear comprising a thin disposable panty shape fabric defining a front portion, a back portion and an intermediate crotch portion there between, to said crotch portion a padded insert is secured in superposed relation forming a padded crotch surface, and the above mentioned front and back portions are attached by a removable attachment means.

None of the above inventions and patents, taken either singly or in combination, are seen to disclose a wearable sanitary napkin/panty liner.

Further, none of this prior art suggests the present inventive combination of component elements arranged and configured for the efficient solution of this problem as disclosed and claimed herein. Prior devices do not provide the benefits of the present invention which achieves its intended purposes, objectives and advantages over the prior art devices through a new, useful and unobvious combination of component elements, through no increase in the number of functioning parts, at a minimum cost and through the utilization of only readily available materials and conventional components.

### SUMMARY OF THE INVENTION

The present invention solves the problems of the prior art by providing a novel wearable sanitary napkin/panty liner including a pocket for placing a sanitary napkin in a fixed position.

The primary intent of the present invention is to provide a wearable sanitary napkin that can be properly positioned and maintained by the user.

Another intent of the present invention is to provide a sanitary napkin supporting panty liner which is comfortable and will permit the wearer to move into different postures without causing the napkin to shift in position.

Another intent of the present invention is to provide a wearable sanitary napkin/panty liner having wearable section further comprising an upper side, a lower side a front surface and an inner surface, pocket located at the inner surface of the wearable section, waist band located at the upper side of the wearable section and the sanitary napkin is placed within the pocket located at the inner surface of the wearable section and the pocket is permanently sealed.

Another intent of the present invention is to provide a disposable absorbent panty liner that can be properly positioned and maintained the sanitary napkin by the user.

Another intent of the present invention is to provide an easy to carry and easy to change wearable sanitary napkin.

Each intent of the present invention provides an advantage of disposable, wearable sanitary napkin.

Each intent of the present invention provides an advantage of sanitary napkin supporting panty liner.

Each intent of the present invention provides an advantage of an easy to carry and easy to change wearable sanitary napkin.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty which characterize the invention, are pointed out with particularity in the disclosure. For a better understanding of the invention, its operating advantages and the specific objects attained by its uses, reference should be had to the accompanying drawings and descriptive matter in which there are illustrated preferred embodiments of the invention.

Additional features and advantages of the invention will be set forth in the detailed description which follows, and in part will be readily apparent to those skilled in the art from that description or recognized by practicing the invention as described herein, including the detailed description which follows, the claims, as well as the appended drawings.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 shows the front view of a wearable sanitary napkin/panty liner in an embodiment of the present invention; and

FIG. 2 shows the inside view of a wearable sanitary napkin/panty liner in an embodiment of the present invention.

### DETAILED DESCRIPTION OF THE INVENTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural and logical changes may be made without departing from the spirit and scope of the present invention. The following detailed description is, therefore, not to be taken in a limiting sense, and the scope of the present invention is defined by the appended claims and their equivalents.

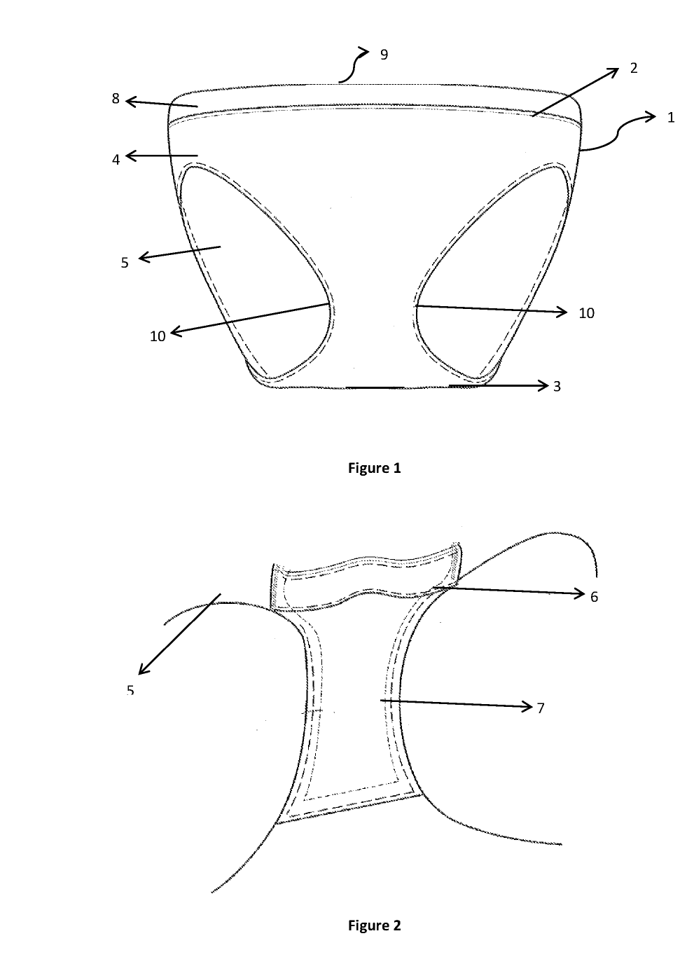

Referring now to the drawings, wherein like reference characters designate identical, or corresponding parts throughout the figures, and in particular FIGS. 1 & 2 show various embodiments of the wearable sanitary napkin/panty liner in accordance with the present invention.

FIG. 1 shows the front view of the wearable sanitary napkin/panty liner including a wearable section 01 with an upper side 02, a lower side 03, a front surface 04 & an inner surface 05, waist band 08, hole on the upper side 09 and two holes 10 at the lower side.

FIG. 2 shows the inside view of a wearable sanitary napkin/panty liner including the inner surface 05, a pocket 06 and sanitary napkin 07.

The present invention generally provides a wearable sanitary napkin/panty liner that can be properly positioned and maintained by the user, including the wearable section 01, the sanitary napkin 07, the pocket 06 for placing the sanitary napkin 07 and the waist band 08.

In an embodiment the present invention provides a wearable sanitary napkin/panty liner made of biodegradable waterproof material, including the wearable section 01, the sanitary napkin 07, the pocket 06 for placing the sanitary napkin 07 and the waist band 08. The wearable section 01 further includes the upper side 02, the lower side 03, the front surface 04 and the inner surface 05. Within the wearable section 01 a hole 09 in the upper side 02 for passing a trunk of a woman's body there through and two holes 10 in the lower side 03 for passing two thighs of the woman's body.

In another embodiment the present invention provides a wearable sanitary napkin/panty liner made of absorbent cotton, including the wearable section 01, the sanitary napkin 07, the pocket 06 for placing the sanitary napkin 07 and the waist band 08. The wearable section 01 further includes the upper side 02, the lower side 03, the front surface 04 and the inner surface 05. Within the wearable section 01 a hole 09 in the upper side 02 for passing a trunk of a woman's body there through and two holes 10 in the lower side 03 for passing two thighs of the woman's body. Also the pocket 06 for placing the sanitary napkin 07 is made of thin permeable membrane and located at the inner surface 05 of the wearable section 01.

In another embodiment the present invention provides a wearable sanitary napkin/panty liner made of absorbent, biodegradable cotton, including the wearable section 01, the sanitary napkin 07 and the pocket 06 for placing the sanitary napkin 07. In this particular embodiment the wearable section 01 is side open and the pocket 06 for placing the sanitary napkin 07 is made of thin permeable membrane and located at the inner surface 05 of the wearable section 01.

It is to be understood that the above description is intended to be illustrative, and not restrictive. For example, the above-discussed embodiments may be used in combination with each other. Many other embodiments will be apparent to those of skill in the art upon reviewing the above description.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention.

What is claimed is:

1. A disposable, wearable sanitary napkin/panty liner comprising:
a) a wearable section further comprising an upper side, a lower side a front surface and an inner surface;
b) a pocket located at the inner surface of the wearable section;
c) a sanitary napkin; and
d) a waist band located at the upper side of the wearable section.

2. A disposable, wearable sanitary napkin/panty liner according to claim 1, wherein the wearable section comprising a hole in the upper side for passing a trunk of a woman's body there through and two holes in the lower side for passing two thighs of the woman's body.

3. A disposable, wearable sanitary napkin/panty liner according to claim 1, wherein the wearable section is made of biodegradable cotton.

4. A disposable, wearable sanitary napkin/panty liner according to claim 1, wherein the inner surface is made of biodegradable absorbent material.

5. A disposable, wearable sanitary napkin/panty liner according to claim 1, wherein the pocket is made of thin permeable material.

6. A disposable, wearable sanitary napkin/panty liner according to claim 1, wherein the sanitary napkin is placed within the pocket located at the inner surface of the wearable section and the pocket is permanently sealed.
